(19) United States
(12) Patent Application Publication
(10) Pub. No.: US 2026/0345912 A1
(43) Pub. Date: Nov. 5, 2026

(54) SYRINGE SYSTEM WITH DEFINED MAXIMUM DOSAGE CAPACITY

(71) Applicant: Becton, Dickinson and Company, Franklin Lakes, NJ (US)

(72) Inventors: Shishir Prasad, Ramsey, NJ (US); Karthik MR, Bengaluru (IN)

(73) Assignee: Becton, Dickinson and Company, Franklin Lakes, NJ (US)

(21) Appl. No.: 19/194,346

(22) Filed: Apr. 30, 2025

(51) Int. Cl. A61M 5/315; A61M 5/31

(52) U.S. Cl. CPC A61M 5/31505; A61M 5/31528; A61M 5/31536; A61M 2005/3114; A61M 2005/31508; A61M 2005/3154

(57) ABSTRACT

A syringe system restricts aspiration capacity of a medical syringe to a defined maximum volume capacity or dosage within the syringe cavity of the syringe barrel. A plunger retraction-stop, coupled to the syringe, prevents additional aspiration of medicinal fluid within the syringe cavity by blocking syringe plunger retraction beyond a designated maximum dosage volume. The plunger retraction-stop includes a stop tip oriented within the syringe cavity between the internal barrel wall and the syringe plunger, intermediate the proximal and distal ends of the plunger. The stop tip blocks further retraction of the plunger when it is in engaging contact with the plunger. In some embodiments of this syringe system, maximum dosage volume is selectively variable, prior to drug administration, while in other embodiments it is fixed during syringe manufacture. Advantageously, use of this syringe system, with its pre-set maximum dosage volume, reduces likelihood of drug overdosage to patients.

### TECHNICAL FIELD

Aspects of the present disclosure generally relate to a syringe assembly for administering medicinal fluids to a patient. More particularly, aspects of the present disclosure are directed to a syringe system that restricts aspiration capacity of a medical syringe to a defined maximum volume capacity or dosage.

### BACKGROUND

In the past, injected drugs were typically administered in licensed clinical environments, such as medical offices, clinics or hospitals. Trained medical professionals performed the patient injections, whether injection delivery was intra-venous, subcutaneous or intramuscular. Medical professionals follow injection protocols, including procedures for assuring administration of correct drug dosage, to reduce risk of drug overdosage.

Pharmaceutical research has led to development of an increasing number of drugs for treatment of various medical conditions, such as arthritis, cholesterol management, asthma, deep vein thrombosis, cardiovascular diseases, osteoporosis, autoimmune conditions, infections, infertility, diabetes, and weight loss. Many of these newly developed drugs are administered by subcutaneous or intramuscular injection. Medical regulatory authorities are permitting self-injection of drugs, directly administered by the patient or by a family caregiver in a non-clinical environment. Most patients and home care givers who are injecting medications outside of professionally supervised clinical facilities are not formally trained in medically recognized injection protocols. Overdosing of self-administered drugs, due to lack of sufficient medical training, ignorance or carelessness, poses a significant risk to patients. Some self-injected medications are prepackaged in specialized delivery devices that only administer the prescribed dose in each injection cycle, which reduces patient overdose risk.

However, many self-injected drugs are not packaged in specialized delivery devices that limit maximum drug injection dosage. Many of these drugs are provided to patients in multi-dose vials. The patient or caregiver must aspirate each individual drug dose from the multi-dose vial with a medical syringe, then perform the injection. Typical medical syringes provided for self-injection are capable of aspirating a range of liquid volumes that are less than or exceed the patient's prescribed dosage volume. Thus, one potential cause of drug overdosing is aspiration of an incorrect, greater volume of medication within a dosing syringe—whether by over-retracting the syringe plunger or by misreading the dosing scale on the syringe barrel or by incorrectly interpreting dosing units of measure.

### SUMMARY

A syringe system restricts aspiration capacity of a medical syringe to a defined maximum volume capacity or dosage within the syringe cavity of the syringe barrel. This effectively pre-sets the maximum deliverable dosage of the syringe. Limiting maximum syringe capacity reduces likelihood of drug overdosage to patients, especially in home-injection, or self-injection and other non-clinical environments, where the injection administrator is not a licensed medical professional who has extensive training in drug injection protocols. In syringe systems of the present disclosure, a plunger retraction-stop, coupled to the syringe, incorporates a stop tip that is oriented within the retraction path of the syringe plunger. The retraction-stop prevents additional aspiration of medicinal fluid within the syringe cavity by blocking syringe plunger retraction beyond what is needed to establish a designated maximum dosage volume. The stop tip is oriented within the syringe cavity between the internal barrel wall and the syringe plunger, intermediate the proximal and distal ends of the plunger. During syringe plunger retraction, when the stop tip is in engaging contact with the plunger, it blocks further retraction of the plunger, thereby limiting maximum volume capacity of the syringe for drug aspiration. In some embodiments of this syringe system, maximum dosage volume is selectively variable, while in other embodiments it is fixed during syringe manufacture.

One aspect of the present disclosure pertains to an aspiration capacity-restricted syringe system, which includes a syringe and a plunger-retraction stop. The syringe has a syringe barrel with a distal end that defines an outlet, an open proximal end, and an internal barrel wall that defines a syringe cavity therein between the distal and proximal ends thereof; and a syringe plunger inserted within the syringe cavity. The syringe plunger has a distal axial end and a peripheral circumferential surface that is in fluid sealing, abutting contact with the internal barrel wall. The plunger is translatable along a plunger retraction path within the syringe cavity, to vary selectively delivery volume within the syringe cavity between the distal end of the syringe barrel and the distal axial end of the plunger by advancement or retraction of the plunger. The plunger retraction-stop is coupled to the syringe. The plunger retraction-stop includes a stop tip oriented within the syringe cavity between the internal barrel wall and the syringe plunger, intermediate the proximal and distal ends of the plunger. The stop tip intersects the retraction path of the syringe plunger and blocks further retraction of the plunger when it is in engaging contact with the plunger.

Another aspect of the present disclosure pertains to an aspiration capacity-restricted syringe system, which includes a syringe and a plunger-retraction stop. The syringe has a syringe barrel with a distal end that defines an outlet, an open proximal end, and an internal barrel wall that defines a syringe cavity therein between the distal and proximal ends thereof, a syringe flange on the proximal end of the syringe barrel, and a syringe plunger inserted within the syringe cavity. The syringe plunger has a distal axial end and a peripheral circumferential surface that is in fluid sealing, abutting contact with the internal barrel wall. The plunger is translatable along a plunger retraction path within the syringe cavity, to vary selectively delivery volume within the syringe cavity between the distal end of the syringe barrel and the distal axial end of the plunger by advancement or retraction of the plunger. The plunger retraction-stop is coupled to the syringe flange.

In some embodiments, the maximum dosage regulated by the retraction-stop is fixed during manufacture of the syringe system. In other embodiments, the syringe's maximum dosage is selectively variable by repositioning the stop tip of the retraction-stop. Exemplary additional embodiments of fixed and selectively variable, maximum dosage, syringe systems of the present disclosure are as follows.

In some embodiments of the present disclosure, the plunger retraction-stop further includes a plunger retraction-stop housing coupled to the syringe barrel, such as to the syringe flange of the syringe barrel and defines a rack channel that is oriented in alignment with the proximal open end of the syringe barrel. An elongated, translatable rack is retained within the rack channel. The rack defines a first helical thread pattern intermediate its proximal and distal ends, with the distal end thereof defining the stop tip. The distal end of the rack and its stop tip are oriented within the syringe cavity between the internal barrel wall and the syringe plunger, intermediate the proximal and distal ends of the plunger. A rotatable regulating wheel is coupled to the plunger retraction-stop housing. The regulating wheel defines a second helical thread that is in mating engagement with the first thread pattern, so that rotation of the regulating wheel advances or retracts the stop tip of the rack within the syringe cavity. The maximum capacity of the syringe cavity is selectively regulated by rotation of the regulating wheel.

In some embodiments, a plunger retraction-stop housing coupled to the syringe barrel, such as to the syringe flange of the syringe barrel and defines a rack channel that is oriented in alignment with the proximal open end of the syringe barrel. An elongated, translatable rack is retained within the rack channel. The rack defines a first tooth pattern that is oriented intermediate its proximal and distal ends, with the distal end thereof defining the stop tip. The distal end of the rack and its stop tip are oriented within the syringe cavity between the internal barrel wall and the syringe plunger, intermediate the proximal and distal ends of the plunger. A pinion profile, rotatable regulating wheel is coupled to the plunger retraction-stop housing. The pinion regulating wheel defines a second tooth pattern that is in mating engagement with the first tooth pattern, so that rotation of the pinion regulating wheel advances or retracts the stop tip of the rack within the syringe cavity. The maximum capacity of the syringe cavity is selectively regulated by rotation of the pinion regulating wheel.

In some embodiments of the present disclosure, the plunger retraction-stop further includes a plunger retraction-stop housing coupled to the syringe barrel, such as to the syringe flange of the syringe barrel and defines a rack channel that is oriented in alignment with the proximal open end of the syringe barrel. An elongated, translatable rack is retained within the rack channel. The rack defines a first hypoid-gear tooth pattern that is oriented intermediate its proximal and distal ends, with the distal end thereof defining the stop tip. A rotatable regulating wheel is coupled to the plunger retraction-stop housing, the regulating wheel defines a second hypoid-gear tooth pattern on an outer circumferential surface thereof that is in mating engagement with the first hypoid-gear tooth pattern, so that rotation of the regulating wheel advances or retracts the stop tip of the rack within the syringe cavity. The maximum capacity of the syringe cavity is selectively regulated by rotation of the regulating wheel.

In some embodiments of the present disclosure, the plunger retraction-stop further includes a plunger retraction-stop housing having a distal end inserted within the open proximal end of the syringe barrel. The housing defines a plunger channel therethrough that captures a shaft of the translatable syringe plunger therein. An elongated stop sleeve is oriented within the syringe cavity between the internal barrel wall and the plunger, circumscribing at least a portion of the plunger shaft. The stop sleeve is interposed between the distal end of the plunger retraction-stop housing and the distal axial end of the syringe plunger, with a distal end of the stop sleeve defining the stop tip. In some embodiments, the stop sleeve and stop tip are a projection that is integrally formed within the retraction-stop housing. The maximum capacity of the syringe cavity is regulated by the overall axial length of the elongated stop sleeve.

In some embodiments of the present disclosure, the plunger retraction-stop further includes a plunger retraction-stop housing is coupled to the syringe barrel, such as to the syringe flange of the syringe barrel. The housing oriented in alignment with the proximal open end of the syringe barrel. A cantilever-like pawl is coupled to the plunger retraction-stop housing; a distal tip of the pawl defines the stop tip that projects into the syringe cavity. The syringe plunger defines an engagement surface, in biased, abutting contact with the stop tip of the pawl. The plunger also defines a notch formed in the plunger engagement surface. Mutual engagement and capture of the stop tip within the notch formed in the plunger engagement surface blocks further retraction of the plunger. Maximum capacity of the syringe cavity is selectively regulated by axial location of the notch in the plunger engagement surface, such as during manufacture of the syringe system.

Yet another aspect of the present disclosure is directed to a method for restricting aspiration capacity of a medical syringe having a syringe having: a syringe barrel, a syringe plunger inserted within the syringe barrel that is retractable along a retraction path, and a plunger retraction-stop coupled to the syringe. The plunger retraction-stop includes a stop tip oriented within the syringe barrel and intersecting the plunger retraction path. In this method, the stop tip is oriented in the syringe plunger retraction path to block retraction of the plunger beyond a position corresponding to the syringe's aspiration capacity of a prescribed drug dose. The prescribed drug dose is aspirated into the syringe barrel by retracting the syringe plunger. Further plunger retraction is blocked when it is in engaging contact with the stop tip of the plunger retraction-stop. When further plunger retraction is blocked, the syringe cannot aspirate any additional drug volume into the syringe barrel beyond the prescribed drug dose. Thus, use of the syringe system of this disclosure reduces chance of patient drug overdose.

### BRIEF DESCRIPTION OF THE DRAWINGS

Exemplary embodiments of the disclosure are further described in the following detailed description in conjunction with the accompanying drawings, in which:

FIG. 1 is an isometric view of an embodiment of a syringe system of the present disclosure;

FIG. 2 is a partial, axial cross section of the syringe system of FIG. 1;

FIG. 3 is another partial, axial cross section of the syringe system of FIG. 1;

FIG. 4 is a partial exploded view of the syringe system of FIG. 1;

FIG. 5 is an isometric view of another embodiment of a syringe system of the present disclosure;

FIG. 6 is an isometric view of an alternative embodiment of a syringe system of the present disclosure;

FIGS. 7 and 7A are isometric views of an additional embodiment of a syringe system of the present disclosure, which includes a plug-type housing stop;

FIGS. 8-11 are isometric end views of alternative embodiments of plug-type housing stop components of the syringe system of FIG. 7;

FIG. 12 is an isometric view of an additional embodiment of a syringe system of the present disclosure;

FIG. 13 is an isometric view of an additional embodiment of a syringe system of the present disclosure; and

FIG. 14 is a partial, axial cross section of the syringe system of FIG. 13.

To facilitate understanding, identical reference numerals have been used, where possible, to designate identical elements that are common to the figures. The figures are not drawn to scale.

### DETAILED DESCRIPTION

Various embodiments of syringe systems disclosed herein restrict passively aspiration capacity of a medical syringe to a defined maximum volume capacity corresponding to that of the prescribed drug or other medicinal fluid that is to be administered to a patient. Aspiration capacity restriction is pre-set during syringe manufacture or selectively prior to injection administration. Limiting maximum syringe capacity reduces likelihood of drug overdosage to patients, especially in home treatment and other non-clinical environments, where the dosing administrator is not a licensed medical professional who has extensive training in intravenous drug administration protocols. Each of the syringe system embodiments of this disclosure incorporates a plunger retraction-stop that prevents additional aspiration of medicinal fluid within the syringe cavity by blocking syringe plunger retraction beyond what is needed to establish a designated maximum dosage volume. The plunger retraction-stop is coupled to the syringe, for example by circumscribing the exterior of the syringe barrel, or by being attached to a flange of the syringe barrel, or by being inserted as a plug into the open end of the syringe barrel cavity. The plunger retraction-stop incorporates a stop tip that is oriented within the retraction path of the syringe plunger. More specifically, the stop tip is oriented within the syringe cavity between the internal barrel wall and the syringe plunger, intermediate the proximal and distal ends of the syringe plunger. During syringe plunger retraction, when the stop tip is in engaging contact with the plunger, it blocks further retraction of the plunger, thereby limiting maximum volume capacity of the syringe, corresponding to the prescribed drug dose. In some embodiments of this syringe system, maximum dosage volume is selectively variable, while in other embodiments it is fixed during syringe manufacture. Plunger retraction-stop embodiments of the present disclosure are adaptable for use with existing commercially available, variable aspiration volume syringes.

Before describing several exemplary embodiments of the disclosure, it is to be understood that the disclosure is not limited to the details of construction or process steps set forth in the following description. The disclosure is capable of other embodiments and of being practiced or being conducted in many ways.

In this disclosure, a convention is followed wherein the distal end of the device is the end closest to a patient, e.g., for delivery of one or more drugs to the patient, and the proximal end of the device is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, I.V. tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a syringe discharge tip and can interlock and connect to the female end that is incorporated within a proximal end of a needle hub of a drug dispensing needle or filling needle for aspirating a drug from a drug vial into a syringe. Luer connector female ends are commonly located on vascular access devices (VADs).

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “wall”, “top”, “side”, “bottom” and others are used throughout this specification to facilitate understanding, it is not intended to limit any components that can be used in combinations or individually or to require specific spatial orientations, to implement various aspects of the embodiments of the present disclosure.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

In an exemplary implementation of the embodiments of present disclosure, a barrel of a syringe includes a distal end having a needleless connection. In one or more embodiments, the needleless connection includes at least one thread and other features in any and all combinations allowing it to interface with a corresponding thread or plurality of threads of a corresponding connector.

According to further exemplary implementations of the embodiments of the present disclosure, configuration of structural elements making up the needleless connector include a collar protruding from the distal end of the barrel, the collar comprising at least one thread to connect to the corresponding thread or plurality of threads of a corresponding connector or needle hub.

According to still further exemplary implementations of the embodiments of the present disclosure, the collar or the needleless connector generally may bend or elastically deform in order to allow better interference fit compliance with corresponding connectors.

According to still further exemplary implementations of the embodiments of the present disclosure, the needleless connector may comprise female threads that are sized and have a thread pattern that will engage with a standard ISO594-2 type of male fitting and/or a male threads that are sized and have a thread pattern that will engage with a standard ISO594-2 type of female fitting. An example of an ISO594-2 type of fitting is a Q-style fitting.

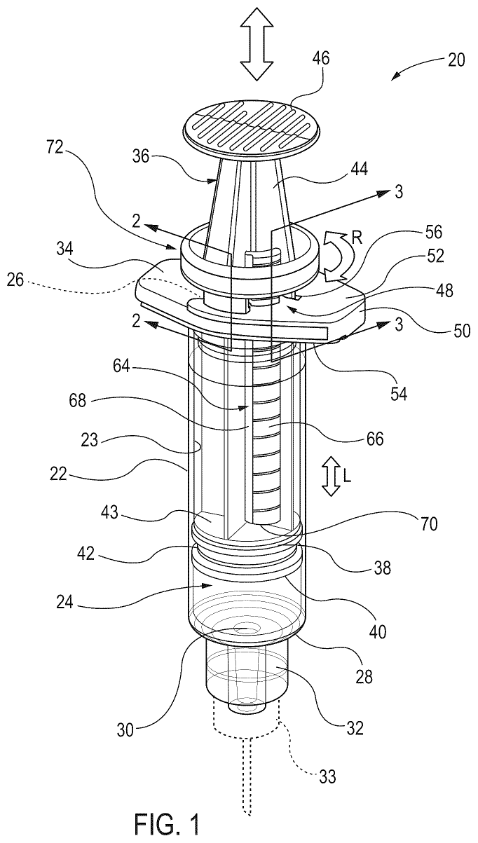

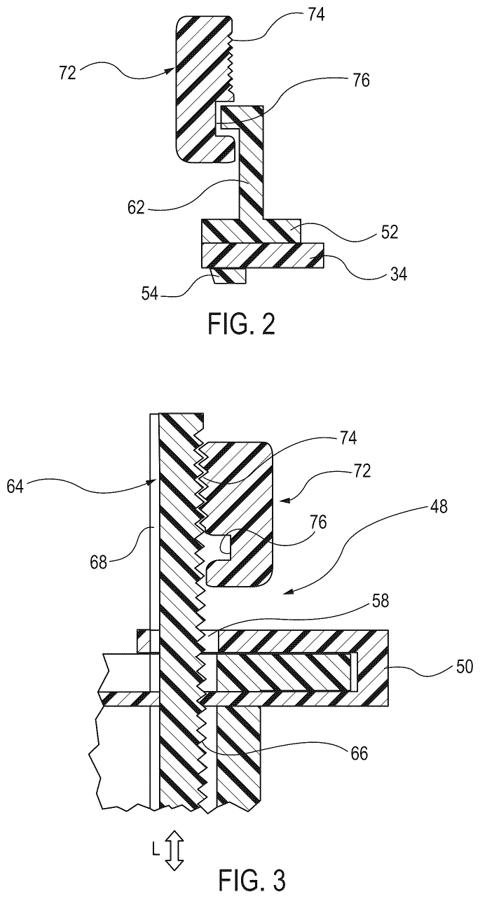

The following non-limiting examples demonstrate principles according to one or more embodiments of the disclosure. FIGS. 1-4 depict a first embodiment of a medical injection device or syringe system 20, with a barrel 22 that defines an internal barrel wall 23 and therein a barrel cavity 24 with a proximal open end 26 and a barrel end or tip 28 on a distal end of the barrel that terminates in an outlet 30. In some embodiments, the barrel tip 28 comprises a Luer connector 32. A needle hub and needle 33 (shown in dashed lines), are coupled to the Luer connector 32, in fluid communication with the outlet 30. The syringe barrel 22 incorporates a syringe flange 34 at its proximal end. A syringe plunger 36 is inserted in the proximal open end 26 of the barrel and is translatable along a translation path within the barrel cavity 24. The distal end of the plunger 36 has a plunger stopper 38. As a component of the plunger 36, the plunger stopper 38 defines a distal axial end 40 and a peripheral circumferential surface 42 of the plunger 36 that are in fluid sealing, abutting contact with the internal barrel wall 23. In some embodiments, the plunger and plunger stopper are integrally formed. A plunger contact surface 43 is oriented on plunger 36, facing the proximal open end 26 of the syringe barrel. A dose of a drug or other medical fluid is contained in the barrel cavity 24 between the barrel end or tip 28 and the distal axial end 40 of the plunger stopper 38. As is common with medical syringes, such as the syringe system 20, the barrel cavity 24, barrel tip 28, outlet 30 Luer connector 32 are in fluid communication with each other for administration of medicinal fluids through the needle tip 33 into a patient at a desired injection delivery site. Drugs are aspirated into and dispensed from the barrel cavity by finger manipulation and translation of the plunger shaft 44 and plunger tab 46 of the plunger 36 external the barrel cavity 24, while grasping the syringe flange 34. Exemplary common, self-administered injections are subcutaneous or intramuscular injections to abdominal and/or muscle tissue sites. The syringe barrel 22 and the plunger 36 are capable of aspirating and dispensing a plurality of drug dosage volumes, depending upon the relative axial position of the distal axial end 40 of the plunger stopper 38. Syringe barrels commonly incorporate indicia lines that correspond to liquid volume contained within the barrel cavity 24. A drug overdose can occur when the plunger 36 is retracted during drug aspiration beyond the indicia line corresponding to the prescribed drug dose. For example, if a syringe has a 70 ml aspiration volume capacity and the drug dose is 40 ml, retracting the plunger beyond the syringe's 40 ml indicia line will exceed the prescribed drug dose volume.

The syringe system 20 includes a plunger retraction-stop mechanism 48, which blocks retraction of the plunger 36 and corresponding increase of barrel cavity 24 volume, more than that of the prescribed drug dose. The retraction stop-mechanism 48 is calibrated to correspond syringe barrel cavity 24 volume with the prescribed drug dose volume. For example, if the prescribed drug dose is 40 ml, the retraction-stop mechanism 48 is also calibrated to block plunger retraction beyond a corresponding 40 ml volume in the syringe cavity 24.

In the embodiment of FIGS. 1-4, the plunger retraction-stop mechanism 48 comprises a plunger retraction-stop housing 50 with axially separated, proximal 52 and distal 54 retention plates for capturing the syringe flange 34 therebetween. An anti-clocking channel 56 captures an outboard edge of the cruciform-shaped plunger shaft 44 therein. Housing 50 incorporates a rack channel 58 that is oriented in alignment with the proximal open end 26 of the syringe barrel 22, and a pair of first 60 and second 62 retention flanges.

The plunger retraction-stop mechanism 48 further comprises an elongated, translatable rack 64, having axial central axis that is parallel to an axial central axis of the syringe plunger 36 and a sector-shaped cross section, with its outer circumferential radius of the sector defining a first helical thread pattern 66 intermediate its proximal and distal ends and outboard radial edges defining anti-rotation steps 68 that are parallel to the axial central axis of the rack. The rack 64 is slidably captured within the rack channel 58, the latter having a plan profile, perpendicular to the central axis of the plunger 36, which conforms to the cross section of the rack 64. The rack 64 is oriented within and projects into the syringe cavity barrel cavity 24, between the internal wall 23 of the barrel 22 and the syringe plunger 36 (i.e., between the plunger shaft 44 and the syringe barrel), intermediate the proximal and distal ends of the plunger. A distal end of the rack 64 forms a stop tip 70 that projects into the syringe cavity 24 in opposed orientation with the plunger contact surface 43 of the plunger 36. The stop tip 70 intersects the retraction path of the syringe plunger 36. Plunger 36 retraction is blocked once the plunger 36 is retracted a sufficient axial distance to cause the plunger contact surface 43 to contact the stop tip 70. Axial translation and positioning of the stop tip 70 regulates maximum retraction of the plunger 36, thereby regulating and pre-setting maximum volume of medicinal fluid that can be aspirated into the plunger barrel cavity 24.

In the syringe system 20 embodiment of FIGS. 1-4, axial position of the stop tip 70 is selectively variable by interaction of the translatable rack 64 and an annular-shaped, rotatable regulating wheel 72, the latter of which defines a rotational axis that is coaxial with the axial central axis of the syringe plunger 36, and that is rotatively coupled to the plunger retraction-stop housing 50. Regulating wheel 72 circumscribes the syringe plunger 36 and the rack 64; it defines a second helical thread pattern 74 on its annular, inner circumferential surface that is in mating engagement with the first helical thread pattern 66 of the rack 64. Selective rotation of regulating wheel 72 converts its rotational motion (i.e., double arrow R in FIG. 1) to rack 64 linear translation (i.e., double arrow L in FIGS. 1 and 3), so that the rack's stop tip 70 advances or retracts within syringe barrel 22. Separation of the respective first thread pattern 66 and the second thread pattern 74 is restrained and prevented by mutual engagement of an annular groove 76, formed on the annular, inner circumferential surface of the regulating wheel 72 with the radially outwardly oriented first 60 and second 62 retention flanges of the plunger retraction-stop housing 50. Interaction of the annular groove 76 and the retention flanges 60, 62 prevent relative axial separation of the regulating wheel 72 from the plunger retraction-stop housing 50. In some embodiments, the annular groove is formed on an exterior circumferential surface of the regulating wheel and the corresponding retention flanges are radially inwardly directed, positioned outboard of the regulating wheel. In some embodiments there is a single retention flange and in other embodiments there are more than two retention flanges.

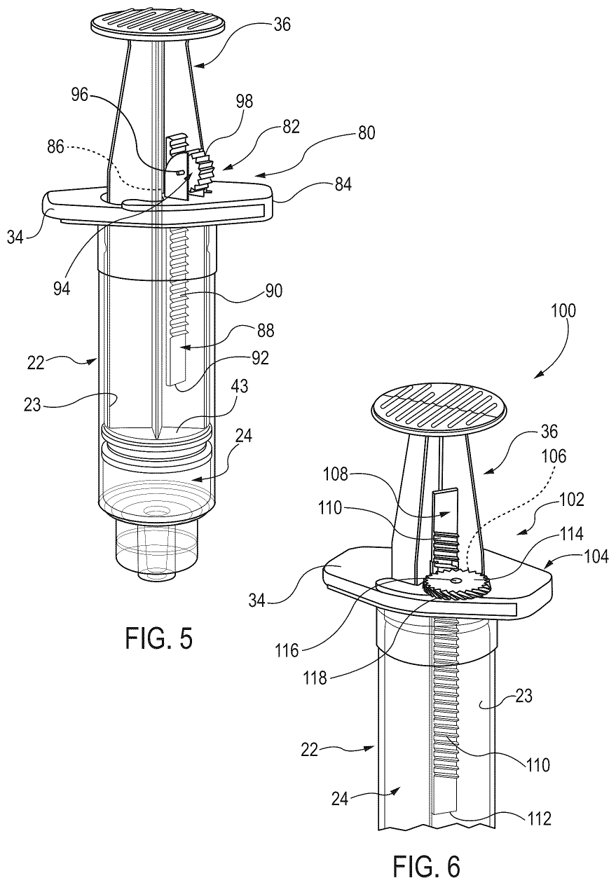

FIGS. 5 and 6 depict other embodiments of syringe systems that utilize plunger retraction-stop mechanisms with rotation-to linear motion conversion elements. As with the embodiment of FIGS. 1-4, these other syringe system embodiments enable variable regulation of stop tip position within the syringe barrel, in order to vary selectively and limit maximum aspiration volume capacity of the syringe. The syringe system embodiments of FIGS. 5 and 6 comprise the same syringe barrel 22 and syringe plunger 36 of the embodiment of FIGS. 1-4, so for brevity, the common structural elements of all of these embodiments are described with the same structural nomenclature and reference numbers.

The syringe system 80 of FIG. 5 incorporates a rack and pinion-type, plunger retraction-stop mechanism 82 comprising a plunger retraction-stop housing 84 coupled to the syringe flange 34 and a rack channel 86 that is oriented in alignment with the proximal open end of the syringe barrel 22 in the same manner as the plunger retraction-stop housing 50 and rack channel 58 of FIG. 1. The plunger retraction-stop mechanism 82 further comprises an elongated, translatable rack 88 retained within the rack channel 86. The rack 88 defines a first tooth pattern 90 that is intermediate its proximal and distal ends, with the distal end thereof defining a stop tip 92. The distal end of the rack 88 and its stop tip 92 are oriented within the syringe barrel cavity 24 between its internal barrel wall 23 and the syringe plunger 36, intermediate the proximal and distal ends of the plunger. The stop tip 92 projects into the syringe cavity 24 in opposed orientation with the plunger contact surface 43 of the plunger 36. The stop tip 92 intersects the retraction path of the syringe plunger 36. Plunger 36 retraction is blocked once the plunger 36 is retracted a sufficient axial distance to cause the plunger contact surface 43 to contact the stop tip 92. Axial translation and positioning of the stop tip 92 regulates maximum retraction of the plunger 36, thereby regulating and pre-setting maximum volume of medicinal fluid that can be aspirated into the plunger barrel cavity 24.

The plunger retraction-stop mechanism 82 further comprises a pinion profile, rotatable regulating wheel 94 that is coupled to the plunger retraction-stop housing 84 by an axle 96. Pinion regulating wheel 94 defines a second tooth pattern 98 that is in mating engagement with the first tooth pattern 90 of the rack 88. Rotation of the pinion regulating wheel 94 advances or retracts the stop tip 92 of the rack 88 within the syringe cavity 24. Maximum capacity of the syringe cavity 24 is selectively regulated by rotation of the pinion regulating wheel 94.

The syringe system 100 FIG. 6 incorporates a hypoid-type rack and gear wheel plunger retraction-stop mechanism 102 comprising a plunger retraction-stop housing 104 coupled to the syringe flange 34 and a rack channel 106 that is oriented in alignment with the proximal open end of the syringe barrel 22 in the same manner as the plunger retraction-stop housing 50 and rack channel 58 of FIG. 1. The plunger retraction-stop mechanism 102 further comprises an elongated, translatable rack 108 retained within the rack channel 106. Rack 108 defines a first hypoid tooth pattern 110 that is intermediate its proximal and distal ends, with the distal end thereof defining a stop tip 112. The distal end of rack 108 and its stop tip 112 are oriented within the syringe barrel cavity 24 between its internal barrel wall 23 and the syringe plunger 36, intermediate the proximal and distal ends of the plunger. The stop tip 112 projects into the syringe cavity 24 in opposed orientation with the plunger contact surface (not shown) of the plunger 36. The stop tip 112 intersects the retraction path of the syringe plunger 36. Plunger 36 retraction is blocked once the plunger 36 is retracted a sufficient axial distance to cause the plunger contact surface to contact the stop tip 112. Axial translation and positioning of the stop tip 112 regulates maximum retraction of the plunger 36, thereby regulating and pre-setting maximum volume of medicinal fluid that can be aspirated into the plunger barrel cavity 24.

The plunger retraction-stop mechanism 102 further comprises a rotatable regulating wheel 114 that is coupled to the plunger retraction-stop housing 104 by an axle 116. Regulating wheel 114 defines a second hypoid tooth pattern 118 that is in mating engagement with the first tooth pattern 110 of rack 108. Rotation of the regulating wheel 114 advances or retracts the stop tip 112 of the rack 108 within the syringe cavity 24. The maximum capacity of the syringe cavity 24 is selectively regulated by rotation of the regulating wheel 114.

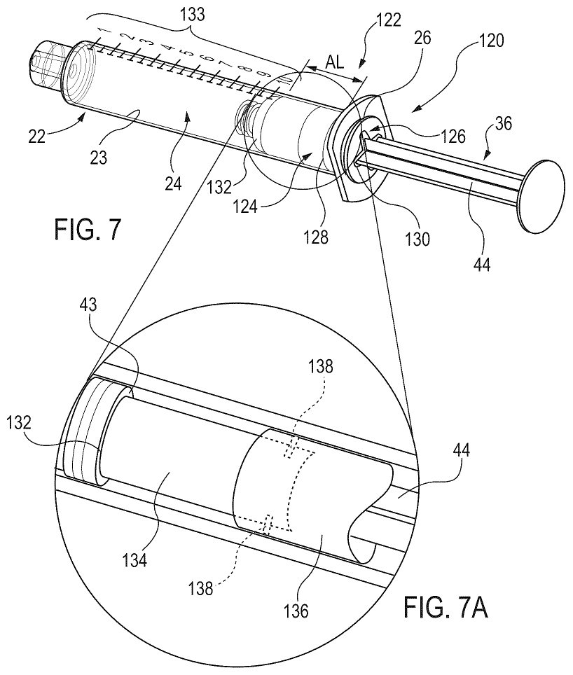

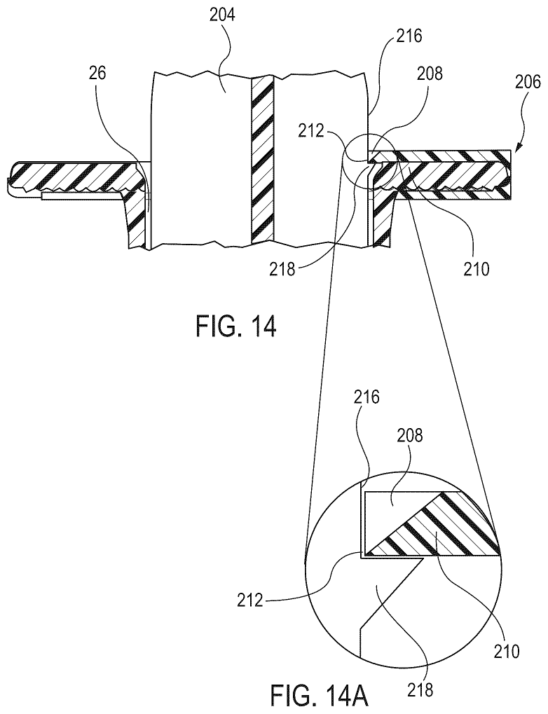

FIGS. 7-14 depict additional embodiments of syringe systems with fixed, maximum capacity plunger retraction-stop mechanisms. In these additional embodiments, regulation of stop tip position within the syringe barrel is pre-set during manufacture. The syringe system embodiments of FIGS. 7-12 comprise the same syringe barrel 22 and syringe plunger 36 of the embodiment of FIGS. 1-4. Similarly, the syringe system embodiments of FIGS. 13 and 14 comprise the same syringe barrel 22 of the embodiment of FIGS. 1-4. For brevity, the common structural elements of all of these embodiments of FIGS. 1-14 are described with the same structural nomenclature and reference numbers.

The syringe system 120 of FIGS. 7-11 incorporates a plunger retraction-stop mechanism 122 with a fixed length stop sleeve 124 that pre-sets the maximum retraction limit of the syringe plunger 36. As a general overview, plunger retraction-stop mechanism 122 comprises a plug-like plunger retraction-stop housing 126 having a distal end 128 inserted within the open proximal end 26 of the syringe barrel 22. Housing 126 defines a plunger channel 130 therethrough that captures therein the shaft 44 of the translatable syringe plunger 36. As shown the elongated stop sleeve 124 is oriented within the syringe cavity 24 between the internal barrel wall 23 and the plunger 36, circumscribing at least a portion of the plunger shaft 44. The stop sleeve 124 is interposed between the distal end 128 of the plunger retraction-stop housing 126 and the plunger contact surface 43 located at the distal axial end of the syringe plunger 36. The plunger stopper 38 is not shown in FIG. 7 but it is shown that figure's enlargement. The distal end of the stop sleeve 124 defines a stop tip 132. As in previously described embodiments, the plunger 36 retraction is blocked once its contact surface 43 abuts the stop tip 132. Thus, maximum dosage capacity of the syringe cavity 24 is regulated by overall axial length of the elongated stop sleeve 124. The syringe system 120 remains capable of aspirating dosages that are less than the maximum dosage capacity by retracting the syringe plunger 36 partially within its maximum translation range to a lesser dosage volume indicated by the indicia dosing indicia 133 on the syringe barrel 22.

To facilitate manufacture of the syringe system 120 with a range of syringe cavity 24 maximum capacities, the stop sleeve 124 further comprises a telescoping sleeve assembly of first 134 and second 136 nested sleeve segments that are rigidly coupled to each other during syringe manufacture, at a desired overall axial length, such as by pins 138. In other embodiments the sleeve segments are joined by means of adhesive or heat welds, or detents, or lanced piercings or mutual threaded engagement. In some embodiments, stop sleeves of different axial lengths are incorporated within the plunger retraction-stop mechanism 122 to regulate the syringe system 120 dosage capacity.

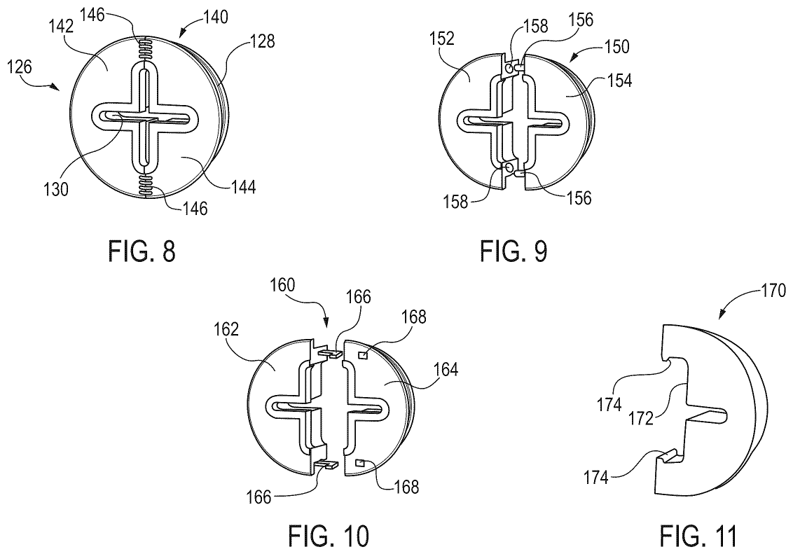

In FIGS. 7 and 8, the plug-like, plunger retraction-stop housing 126 further comprises a necked, split bushing 140, with its necked distal end 128 inserted within the open proximal end 26 of the syringe barrel 22. Bushing 140 has two abutting, first 142 and second 144 bushing halves, which capture the plunger shaft 44 within the collectively defined plunger channel 130. Abutting axial faces of the bushing halves are aligned parallel to the central axis of the plunger shaft 44 and are coupled together by welds 146 or by adhesive.

FIGS. 9-11 depict alternative embodiments of plug-like, plunger retraction-stop housings. In FIG. 9, the plunger retraction-stop housing 150 has abutting, first 152 and second 154 bushing halves that are coupled together with opposed mating pins 156 and pin-receiving apertures 158. In FIG. 10, the plunger retraction-stop housing 160 has abutting, first 162 and second 164 bushing halves that are coupled together with opposed mating snap fasteners 166 and snap-receiving apertures 168. In FIG. 11, plunger retraction-stop housing 170 comprises a half bushing whose plunger channel 172 captures three of the four flange segments of the cruciform-shaped plunger shaft 36 that is shown in FIG. 7. Hooks 174, formed in the housing 170 prevent radial separation of the plunger shaft out of plunger channel 172.

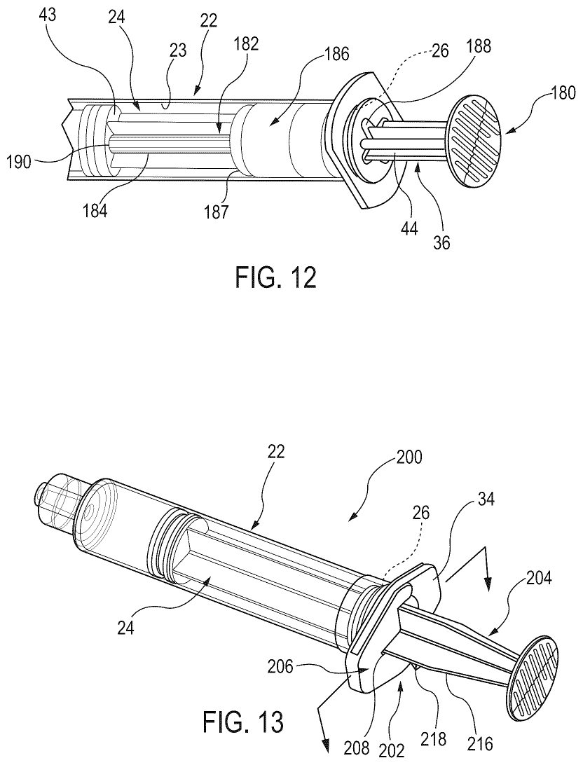

The syringe system 180 of FIG. 12 incorporates a plunger retraction-stop mechanism 182 with a fixed length stop rod 184 that pre-sets the maximum retraction limit of the syringe plunger 36. As a general overview, plunger retraction-stop mechanism 182 comprises a plug-like plunger retraction-stop housing 186 having a distal end 187 inserted within the open proximal end 26 of the syringe barrel 22. Housing 186 defines a plunger channel 188 therethrough that captures therein the shaft 44 of the translatable syringe plunger 36. The elongated stop rod 184 is oriented within the syringe cavity 24 between the internal barrel wall 23 and the plunger 36. Stop rod 184 is coupled to the distal end 187 of the plunger retraction-stop housing 186. The distal end of stop rod 184 defines a stop tip 190. As in previously described embodiments, the plunger 36 retraction is blocked once its contact surface 43 abuts the stop tip 190. Thus, maximum dosage capacity of the syringe cavity 24 is regulated by overall axial length of the elongated stop rod 184.

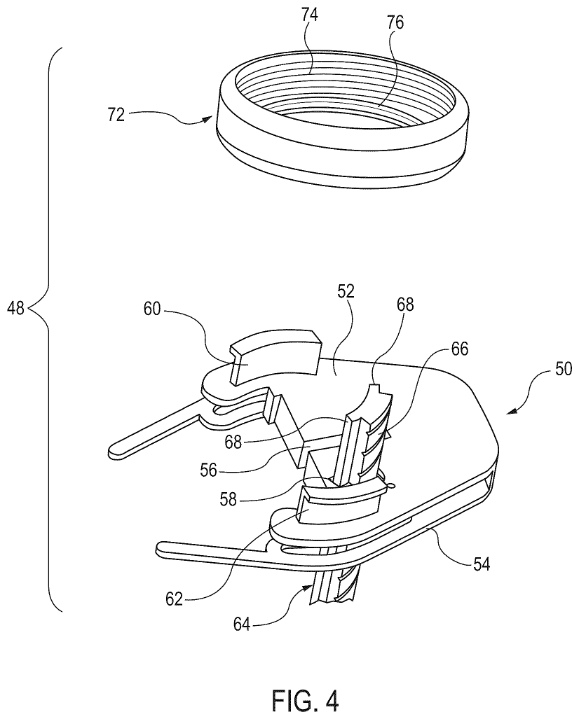

The syringe system 200 embodiment of FIGS. 13 and 14 incorporate a mating pawl and toothed notch-type plunger retraction-stop mechanism 202 that pre-sets the maximum retraction limit of the syringe plunger 204. In FIG. 14, plunger 204 is inserted deeper into the syringe barrel than it is in FIG. 13. The maximum dosage capacity of the syringe system 200 is pre-set during syringe manufacture. A plunger retraction-stop housing 206 is coupled to the syringe flange 34, in alignment with the proximal open end 26 of the syringe barrel 22, similar to the plunger retraction-stop housing 50 of FIG. 1. Housing 206 further comprises an anti-clocking channel 208, similar to anti-clocking channel 56 of FIGS. 1 and 4. A cantilever-like pawl 210 is formed within the anti-clocking channel 208. The distal tip of the pawl 210 defines a stop tip 212; the stop tip projects radially inwardly into the syringe barrel cavity 24. Pawl 210 is integrally formed within the plunger retraction-stop housing 206. In other embodiments, the pawl is separately formed and coupled to the housing. Syringe plunger 204 defines an engagement surface 216 on an outboard edge of one of the cruciform-shaped flanges of the plunger shaft. Engagement surface 216 is in biased, abutting contact with the stop tip 212 of the pawl 210. The engagement surface 216 defines an outwardly projecting, tooth-like notch 218 formed thereupon that projects radially outwardly toward the stop tip 212. In some embodiments, the plunger notch is a depression formed in the engagement surface and the stop tip biases into that notch when they are in axial alignment. In some embodiments, the syringe system 200 has more than one pair of opposing notches and stop tips in its plunger retraction-stop mechanism 202. The stop tip 212 intersects the retraction path of notch 218 of the syringe plunger 204. As shown in FIG. 14, plunger 204 retraction is blocked once it is retracted a sufficient axial distance to cause the notch 218 to contact the stop tip 212 of the pawl 210. The maximum dosage capacity of the syringe cavity 23 is pre-set during syringe system 200 manufacture axial location of the notch 218 along the plunger engagement surface 216. Respective, different dosage capacities for the syringe system 200 require specific, associated syringe plungers with notch locations corresponding to the desired dosage.

Exemplary methods for using one or more of the previously described embodiments of the syringe system are disclosed as follows. A pharmacy or other legal drug-dispenser provides a patient or designated caregiver with one or more doses of a prescribed pharmaceutical and a sufficient quantity of syringes needed to dispense those doses. The patient's dosing administrator prepares the drug for injection, in accordance with prescription instructions, such as by mixing diluent with powdered drug in a multiple dose, drug vial, or the like. The risk of drug overdosing decreases when using one of the syringe system embodiments of this disclosure, so long as the prescribed dose matches the maximum preset or adjusted aspiration capacity of the provided syringe system. For example, where a prescribed drug dose is 40 ml, the pharmacy provides the patient syringes of the present disclosure that are preset or adjusted for a matching 40 ml aspiration maximum capacity.

In some dispensing scenarios, each provided syringe incorporates a preset, maximum capacity, fixed plunger retraction-stop, coupled to the syringe, such as in the embodiments 120, 180 or 200 of FIGS. 7, 7A, 12, 13 and 14. These provided syringe systems prevent additional aspiration of medicinal fluid within the syringe cavity by blocking syringe plunger retraction beyond the preset maximum dosage volume, when the stop tip of the syringe's plunger retraction-stop blocks further retraction of the plunger. While following approved drug injection protocols, the patient's dosing administrator prepares the syringe, aspirates the prescribed dose volume by retracting the plunger to its blocked retraction position, and injects the drug into the patient.

In other dispensing scenarios each provided syringe includes a variable maximum capacity, plunger retraction-stop, coupled to the syringe, such as in the embodiments 20, 80 or 100 of FIGS. 1-6. Prior to dispensing a drug, the regulating wheel of any of these syringe system embodiments is adjusted to match the prescribed drug dose. In some embodiments, the regulating wheel is pre-adjusted during manufacture. In other embodiments, the pharmacy or other dispenser adjusts the regulating wheel prior to transfer to the patient or the dosing administrator. In yet another embodiment, the dosing administrator adjusts the syringe's regulating wheel at home prior to injection of the patient. After adjusting the syringe system's regulating wheel, these provided syringes prevent additional aspiration of medicinal fluid within the syringe cavity by blocking syringe plunger retraction beyond the preset maximum dosage volume, when the stop tip of the syringe's plunger retraction-stop blocks further retraction of the plunger. While following approved drug injection protocols, the patient's dosing administrator prepares the syringe, aspirates the prescribed dose volume by retracting the plunger to its blocked retraction position, and injects the drug into the patient. Advantageously disclosed, variable maximum capacity syringe systems are capable of administering different drug dose volumes. When a patient is prescribed multiple prescriptions of injectable drugs or other medical fluids, each can be dispensed with a single syringe system model by dedicating and adjusting individual syringes to dispense each individual drug prescription dose.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. An aspiration capacity-restricted syringe system, comprising:
a syringe having: a syringe barrel with a distal end that defines an outlet, an open proximal end, and an internal barrel wall that defines a syringe cavity therein between the distal and proximal ends thereof; and a syringe plunger inserted within the syringe cavity, the syringe plunger having a distal axial end and a peripheral circumferential surface that is in fluid sealing, abutting contact with the internal barrel wall, the plunger translatable along a plunger retraction path within the syringe cavity to vary selectively delivery volume within the syringe cavity between the distal end of the syringe barrel and the distal axial end of the plunger by advancement or retraction of the plunger; and
a plunger retraction-stop coupled to the syringe, the plunger retraction-stop including a stop tip oriented within the syringe cavity between the internal barrel wall and the syringe plunger, intermediate the proximal and distal ends of the plunger, the stop tip intersecting the retraction path of the syringe plunger and blocking further retraction of the plunger when it is in engaging contact with the plunger.

2. The aspiration capacity-restricted syringe system of claim 1, the plunger retraction-stop further comprising:
a plunger retraction-stop housing coupled to the syringe barrel, defining a rack channel that is oriented in alignment with the proximal open end of the syringe barrel;
an elongated, translatable rack retained within the rack channel, the rack defining a first helical thread pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a rotatable regulating wheel coupled to the plunger retraction-stop housing, the regulating wheel defining a second helical thread that is in mating engagement with the first thread pattern, so that rotation of the regulating wheel advances or retracts the stop tip of the rack within the syringe cavity;
wherein maximum capacity of the syringe cavity is selectively regulated by rotation of the regulating wheel.

3. The aspiration capacity-restricted syringe system of claim 2, the plunger retraction-stop further comprising:
an annular-shaped regulating wheel circumscribing the syringe plunger and the rack, with the second helical thread pattern and an annular groove oriented on an inner circumferential surface of the regulating wheel; and
at least one flange formed on a proximal surface of the plunger retraction-stop housing that engages within the annular groove, for prevention of axial separation of the regulating wheel from the plunger retraction-stop.

4. The aspiration capacity-restricted syringe system of claim 3, further comprising:
the rack having an axial central axis that is parallel to an axial central axis of the syringe plunger and a sector-shaped cross section, with its outer circumferential radius defining the first thread pattern and at least one radial edge defining an anti-rotation step that is parallel to the axial central axis of the rack;
cross section of the rack channel conforming to that of the rack; and
the regulating wheel having a rotational axis that is coaxial with the axial central axis of the syringe plunger.

5. The aspiration capacity-restricted syringe system of claim 1, the plunger retraction-stop further comprising:
a plunger retraction-stop housing coupled to the syringe barrel, defining a rack channel that is oriented in alignment with the proximal open end of the syringe barrel;
an elongated, translatable rack retained within the rack channel, the rack defining a first tooth pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a pinion profile, rotatable regulating wheel coupled to the plunger retraction-stop housing, the pinion regulating wheel defining a second tooth pattern that is in mating engagement with the first tooth pattern, so that rotation of the pinion regulating wheel advances or retracts the stop tip of the rack within the syringe cavity;
wherein maximum capacity of the syringe cavity is selectively regulated by rotation of the pinion regulating wheel.

6. The aspiration capacity-restricted syringe system of claim 1, the plunger retraction-stop further comprising:
a plunger retraction-stop housing coupled to the syringe barrel, defining a rack channel that is oriented in alignment with the proximal open end of the syringe barrel;
an elongated, translatable rack retained within the rack channel, the rack defining a first hypoid-gear tooth pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a rotatable regulating wheel coupled to the plunger retraction-stop housing, the regulating wheel defining a second hypoid-gear tooth pattern on an outer circumferential surface thereof that is in mating engagement with the first hypoid-gear tooth pattern, so that rotation of the regulating wheel advances or retracts the stop tip of the rack within the syringe cavity;
wherein maximum capacity of the syringe cavity is selectively regulated by rotation of the regulating wheel.

7. The aspiration capacity-restricted syringe system of claim 1, the plunger retraction-stop further comprising:
a plunger retraction-stop housing having a distal end inserted within the open proximal end of the syringe barrel, defining a plunger channel therethrough that captures a shaft of the translatable syringe plunger therein; and
an elongated stop sleeve oriented within the syringe cavity between the internal barrel wall and the plunger, circumscribing at least a portion of the plunger shaft, the stop sleeve interposed between the distal end of the plunger retraction-stop housing and the distal axial end of the syringe plunger, with a distal end of the stop sleeve defining the stop tip;
wherein maximum capacity of the syringe cavity is regulated by overall axial length of the elongated stop sleeve.

8. The aspiration capacity-restricted syringe system of claim 7, the elongated stop sleeve further comprising a telescoping sleeve assembly of at least two nested sleeves that are rigidly coupled to each other at a desired overall axial length.

9. The aspiration capacity-restricted syringe system of claim 7, the plunger retraction-stop housing further comprising a necked, split bushing, with its distal end inserted within the open proximal end of the syringe barrel, the bushing having two abutting halves, abutting faces of the bushing halves aligned parallel to a central axis of the plunger shaft and coupled together by welds, or by adhesive, or by opposed mating pins and pin-receiving apertures, or by opposed mating snap fasteners and snap-receiving apertures.

10. The aspiration capacity-restricted syringe system of claim 1, the plunger retraction-stop further comprising:
a plunger retraction-stop housing coupled to the syringe barrel, which is oriented in alignment with the proximal open end of the syringe barrel;
a cantilever-like pawl, coupled to the plunger retraction-stop housing, a distal tip of the pawl defining the stop tip that projects into the syringe cavity; and
the plunger defining an engagement surface, in biased, abutting contact with the stop tip of the pawl and also defining a notch a notch formed in the plunger engagement surface;
wherein mutual engagement and capture of the stop tip within the notch formed in the plunger engagement surface blocks further retraction of the plunger, and maximum capacity of the syringe cavity is selectively regulated by axial location of the notch in the plunger engagement surface.

11. An aspiration capacity-restricted syringe system, comprising:
a syringe having: a syringe barrel with a distal end that defines an outlet, an open proximal end, and an internal barrel wall that defines a syringe cavity therein between the distal and proximal ends thereof, and a syringe flange on the proximal end of the syringe barrel; and a syringe plunger inserted within the syringe cavity, the syringe plunger having a distal axial end and a peripheral circumferential surface that is in fluid sealing, abutting contact with the internal barrel wall, the plunger translatable along a plunger retraction path within the syringe cavity to vary selectively delivery volume within the syringe cavity between the distal end of the syringe barrel and the distal axial end of the plunger by advancement or retraction of the plunger; and
a plunger retraction-stop coupled to the syringe flange, the plunger retraction-stop including a stop tip oriented within the syringe cavity between the internal barrel wall and the syringe plunger, intermediate the proximal and distal ends of the plunger, the stop tip intersecting the retraction path of the syringe plunger and blocking further retraction of the plunger when it is in engaging contact with the plunger.

12. The aspiration capacity-restricted syringe system of claim 11, the plunger retraction-stop further comprising:
a plunger retraction-stop housing coupled to the syringe flange, defining a rack channel that is oriented in alignment with the proximal open end of the syringe barrel;
an elongated, translatable rack retained within the rack channel, the rack defining a first helical thread pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a rotatable regulating wheel coupled to the plunger retraction-stop housing, the regulating wheel defining a second helical thread that is in mating engagement with the first thread pattern, so that rotation of the regulating wheel advances or retracts the stop tip of the rack within the syringe cavity;
wherein maximum capacity of the syringe cavity is selectively regulated by rotation of the regulating wheel.

13. The aspiration capacity-restricted syringe system of claim 12, the plunger retraction-stop further comprising: an annular-shaped regulating wheel circumscribing the syringe plunger and the rack, with the second helical thread pattern and an annular groove oriented on an inner circumferential surface of the regulating wheel; and at least one flange formed on a proximal surface of the plunger retraction-stop that engages within the annular groove, for prevention of axial separation of the regulating wheel from the plunger retraction-stop.

14. The aspiration capacity-restricted syringe system of claim 13, further comprising:
the rack having an axial central axis that is parallel to an axial central axis of the syringe plunger and a sector-shaped cross section, with its outer circumferential radius defining the first thread pattern and at least one radial edge defining an anti-rotation step that is parallel to the axial central axis of the rack;
cross section of the rack channel conforming to that of the rack; and
the regulating wheel having a rotational axis that is coaxial with the axial central axis of the syringe plunger.

15. The aspiration capacity-restricted syringe system of claim 11, the plunger retraction-stop further comprising:
a plunger retraction-stop housing coupled to the syringe flange, defining a rack channel that is oriented in alignment with the proximal open end of the syringe barrel;
an elongated, translatable rack retained within the rack channel, the rack defining a first tooth pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a pinion profile, rotatable regulating wheel coupled to the plunger retraction-stop housing, the pinion regulating wheel defining a second tooth pattern that is in mating engagement with the first tooth pattern, so that rotation of the pinion regulating wheel advances or retracts the stop tip of the rack within the syringe cavity;
wherein maximum capacity of the syringe cavity is selectively regulated by rotation of the pinion regulating wheel.

16. The aspiration capacity-restricted syringe system of claim 11, the plunger retraction-stop further comprising:
a plunger retraction-stop housing coupled to the syringe flange, defining a rack channel that is oriented in alignment with the proximal open end of the syringe barrel;
an elongated, translatable rack retained within the rack channel, the rack defining a first hypoid-gear tooth pattern intermediate its proximal and distal ends, with the distal end thereof defining the stop tip, the distal end of the rack and its stop tip oriented within the syringe cavity between the internal barrel wall and the syringe plunger, intermediate the proximal and distal ends of the plunger; and
a rotatable regulating wheel coupled to the plunger retraction-stop housing, the regulating wheel defining a second hypoid-gear tooth pattern on an outer circumferential surface thereof that is in mating engagement with the first hypoid-gear tooth pattern, so that rotation of the regulating wheel advances or retracts the stop tip of the rack within the syringe cavity;
wherein maximum capacity of the syringe cavity is selectively regulated by rotation of the regulating wheel.

17. The aspiration capacity-restricted syringe system of claim 11, the plunger retraction-stop further comprising:
a plunger retraction-stop housing having a distal end inserted within the open proximal end of the syringe barrel, defining a plunger channel therethrough that captures therein a shaft of the translatable syringe plunger therein; and
an elongated stop sleeve oriented within the syringe cavity between the internal barrel wall and the plunger, circumscribing at least a portion of the plunger shaft, the stop sleeve interposed between the distal end of the plunger retraction-stop housing and the distal axial end of the syringe plunger, with a distal end of the stop sleeve defining the stop tip;
wherein maximum capacity of the syringe cavity is regulated by overall axial length of the elongated stop sleeve.

18. The aspiration capacity-restricted syringe system of claim 17, the elongated stop sleeve further comprising a telescoping sleeve assembly of at least two nested sleeves that are rigidly coupled to each other at a desired overall axial length.

19. The aspiration capacity-restricted syringe system of claim 11, the plunger retraction-stop further comprising:
a plunger retraction-stop housing coupled to the syringe flange, which is oriented in alignment with the proximal open end of the syringe barrel;
a cantilever-like pawl, coupled to the plunger retraction-stop housing, a distal tip of the pawl defining the stop tip that projects into the syringe cavity; and
the plunger defining an engagement surface, in biased, abutting contact with the stop tip of the pawl and also defining a notch a notch formed in the plunger engagement surface;
wherein mutual engagement and capture of the stop tip within the notch formed in the plunger engagement surface blocks further retraction of the plunger, and maximum capacity of the syringe cavity is selectively regulated by axial location of the notch in the plunger engagement surface.

20. A method for restricting aspiration capacity of a medical syringe, comprising:
providing a syringe having: a syringe barrel; a syringe plunger inserted within the syringe barrel that is retractable along a retraction path; and a plunger retraction-stop coupled to the syringe, the plunger retraction-stop including a stop tip oriented within the syringe barrel and intersecting the plunger retraction path;
orienting the stop tip in the syringe plunger retraction path to block retraction of the plunger beyond a position corresponding to syringe aspiration capacity of a prescribed drug dose;
aspirating the prescribed drug dose into the syringe barrel by retracting the syringe plunger; and
blocking further retraction of the plunger when it is in engaging contact with the stop tip of the plunger retraction-stop, thereby stopping aspiration of additional drug volume into the syringe barrel beyond the prescribed drug dose.
